(19) United States
(12) Patent Application Publication
(10) Pub. No.: US 2026/0335333 A1
(43) Pub. Date: Oct. 29, 2026

(54) METHODS OF TREATING TRAUMATIC ENCYPHALOPATHY SYNDROME

(71) Applicant: Woolsey Pharmaceuticals, Inc., St. Petersburg, FL (US)

(72) Inventors: Thomas MacAllister, Arlingtion, VA (US); Sven Jacobson, New York, NY (US)

(73) Assignee: Woolsey Pharmaceuticals, Inc., St. Petersburg, FL (US)

(21) Appl. No.: 19/484,431

(22) PCT Filed: May 15, 2024
(86) PCT No.: PCT/US2024/029409

§ 371 Date: Nov. 14, 2025

Related U.S. Application Data
(60) Provisional application No. 63/502,676, filed on May 17, 2023.

(51) Int. Cl. A61K 31/551; A61P 25/28; G16H 20/10

(52) U.S. Cl. CPC A61K 31/551; A61P 25/28; G16H 20/10

(57) ABSTRACT

Disclosed is a method of treating traumatic encephalopathy syndrome (TES) in a patient in need thereof by administering a therapeutically effective amount of a rho-kinase inhibitor. In one embodiment, the rho-kinase inhibitor is fasudil.

### BACKGROUND OF THE INVENTION

Chronic traumatic encephalopathy (CTE) is a neurodegenerative disease associated with exposure to repetitive head impacts (RHI) that result in concussive and subconcussive injuries. CTE is pathologically characterized by perivascular tau-immunoreactive neurofibrillary tangles (NFTs) preferentially found at the depths of cortical sulci. At this time, CTE can only be definitively diagnosed upon postmortem analysis of brain tissue to observe the NFTs. CTE diagnoses have been made in players of a variety of sports, including high school, college, and professional American football players; professional and amateur boxers; rugby players; soccer players; college and professional ice hockey players; professional wrestlers; professional baseball players; professional bull riders; and mixed martial arts (MMA) fighters.

While often found in athletes, it has been found in patients with non-sports-related RHI. It can be associated with epilepsy, head banging behavior, domestic abuse and military personal (regularly exposed to blast trauma). While CTE can only be diagnosed definitively post-mortem, it is associated clinically with progressive worsening of cognition, mood, and behavior years to decades after the onset of RHI. The resulting syndrome detectable in life is known as traumatic encephalopathy syndrome (TES).

Currently, there are no effective therapeutic approaches to treating TES. TES is a syndrome found in people with a history of repetitive brain trauma, including symptomatic concussions as well as asymptomatic subconcussive hits to the head that do not cause symptoms. According to recent standards articulated by the National Institutes of Health, a diagnosis of TES requires: (a) substantial exposure to repetitive head impacts (RHIs); (b) cognitive impairment, neurobehavioral dysregulation, or both; and (c) the deficits are not fully accounted for by other disorders and conditions. (Katz et al., 2021) CTE also is progressive. As the condition progresses, further symptoms may include: slurred speech (dysarthria), significant memory problems, parkinsonism—the typical symptoms of Parkinson's disease, including tremor, slow movement and muscle stiffness, rarely, difficulty eating or swallowing (dysphagia).

Because there are no disease modifying treatments, and any treatment of TES is limited to specific symptomatology, there exists a significant unmet need to provide new, approaches to mitigating or reversing TES.

### SUMMARY OF THE INVENTION

Disclosed is the treatment of TES in a patient, including suspected or probable TEC, comprising administering to a patient a rho kinase inhibitor.

In one embodiment, the patient is suffering from a cognitive deficit.

In another embodiment, herein the patient has a history of repetitive head impacts as a result of playing American football.

In a specific embodiment, the patient is 70 years old or younger, preferably 65 years or younger.

In one embodiment, the patient has cavum septum.

In another embodiment, the patient has dementia. In a specific embodiment, the patient has moderate dementia.

In one embodiment, the TES patient is suffering from a neurobehavioral deficit, including but not limited to processing speed, attention; memory, including episodic, working and semantic memory; executive functioning, language and spatial ability; aggression, impulsivity; and depression.

In a specific embodiment, the patient is cognitively impaired.

In a specific embodiment, the rho kinase inhibitor is an isoquinoline derivative, preferably fasudil, a salt, or a derivative thereof.

In one embodiment, treatment of TES continues for at least 6 weeks.

In a specific embodiment, the rho kinase inhibitor is administered in three equal portions throughout the day.

In another specific embodiment, total daily dose is between 70 mg and 240 mg.

In an embodiment, fasudil is administered as fasudil HCl hemihydrate.

In one embodiment, treatment of a TES patient with a therapeutically effective amount of the rho-kinase inhibitor will improve a symptom, reduce the severity of a symptom, or delay progression of symptoms of TES.

In a specific embodiment, treatment of a TES patient with a therapeutically effective amount of the rho-kinase inhibitor such as fasudil will improve cognition. In one embodiment, the improved cognition is an improvement on an accepted evaluation such as the MMSE.

In a specific embodiment, the improvement is an increase in MMSE by at least about 5 points following treatment with the rho-kinase inhibitor. In another embodiment, the MMSE score is increased by at least about 10 points following treatment with the rho-kinase inhibitor

In another embodiment, treatment of a TES patient will improve neurobehavioral symptoms, including but not limited to reducing aggression and depression, outbursts, paranoia, and/or mood swings.

### DETAILED DESCRIPTION OF THE INVENTION

The invention is based on the discovery that rho kinase inhibitors can be sued to treat patients diagnosed with traumatic encephalopathy syndrome (TES). TES is a syndrome found in people with a history of repetitive brain trauma, including symptomatic concussions as well as asymptomatic subconcussive hits to the head that do not cause symptoms. According to recent standards articulated by the National Institutes of Health, a diagnosis of TES requires: (a) substantial exposure to repetitive head impacts (RHIs); (b) cognitive impairment, neurobehavioral dysregulation, or both; and (c) the deficits are not fully accounted for by other disorders and conditions. It has been discovered that, especially in patients no older than 70 years old and having dementia, that rho kinases may be employed to improve cognitive and neurobehavioral function, which are the clinical manifestations of TES.

### ROCK Inhibitors

The inventive methods contemplate the administration of a rho kinase (ROCK) inhibitor in the treatment of a disease or condition. Two mammalian ROCK homologs are known, ROCK1 (aka ROKβ, Rho-kinase β, or p160ROCK) and ROCK2 (aka ROKα) (Nakagawa 1996). In humans, the genes for both ROCK1 and ROCK2 are located on chromosome 18. The two ROCK isoforms share 64% identity in their primary amino acid sequence, whereas the homology in the kinase domain is even higher (92%) (Jacobs 2006; Yamaguchi 2006). Both ROCK isoforms are serine/threonine kinases and have a similar structure.

A large number of pharmacological ROCK inhibitors are known (Feng, LoGrasso, Defert, & Li, 2015). Isoquinoline derivatives are a preferred class of ROCK inhibitors. The isoquinoline derivative fasudil was the first small molecule ROCK inhibitor developed by Asahi Chemical Industry (Tokyo, Japan). The characteristic chemical structure of fasudil consists of an isoquinoline ring, connected via a sulphonyl group to a homopiperazine ring. Fasudil is a potent inhibitor of both ROCK isoforms. In vivo, fasudil is subjected to hepatic metabolism to its active metabolite hydroxyfasudil (aka, M3). Other examples of isoquinoline derived ROCK inhibitors include dimethylfasudil and ripasudil.

Other preferred ROCK inhibitors are based on based on 4-aminopyridine structures. These were first developed by Yoshitomi Pharmaceutical (Uehata et al., 1997) and are exemplified by Y-27632. Still other preferred ROCK inhibitors incude indazole, pyrimidine, pyrrolopyridine, pyrazole, benzimidazole, benzothiazole, benzathiophene, benzamide, aminofurazane, quinazoline, and boron derivatives (Feng et al., 2015). Some exemplary ROCK inhibitors are shown below:

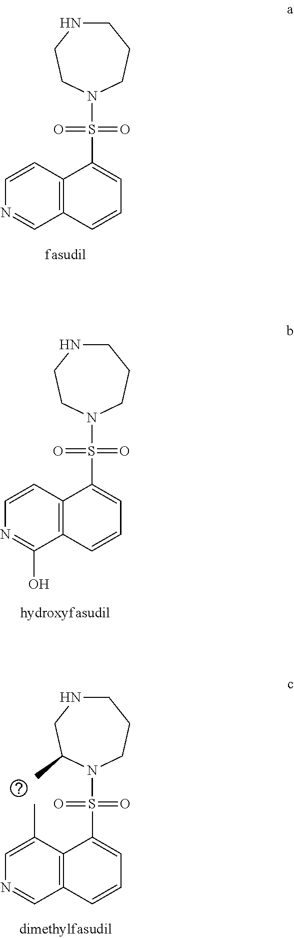 [SMILES: O=S(=O)(c1cccc2cnccc12)N1CCCNCC1 ; O=S(=O)(c1cccc2c(O)nccc12)N1CCCNCC1 ; Cc1cncc2cccc(S(=O)(=O)N3CCCNC[C@@H]3C)c12]

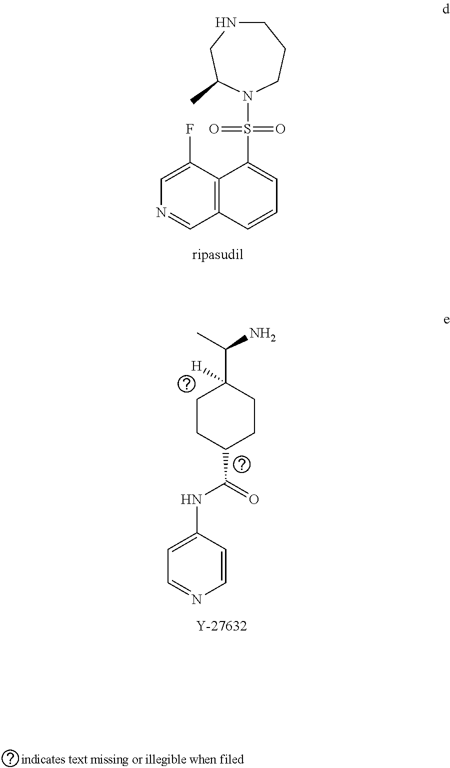

ROCK inhibitors according to the invention may have more selective activity for either ROCK1 or ROCK2 and will usually have varying levels of activity on PKA, PKG, PKC, and MLCK. Some ROCK inhibitors may be highly specific for ROCK1 or ROCK2 and have much lower activity against PKA, PKG, PKC, and MLCK.

A particularly preferred ROCK inhibitor is fasudil. Fasudil may exist as a free base or salt and may be in the form of a hydrate, such as a hemihydrate. Other preferred ROCK inhibitors are netarsudil (AR-13503), belumosudil (KD025, SLx-2119), AT-13148, BA-210, β-Elemene, Chroman 1, DJ4, GSK-576371, GSK429286A, H-1152, LX-7101, RKI-1447, TCS-7001, Thiazovivin, Verosudil, Y-30141, Y-33075, and Y-39983.

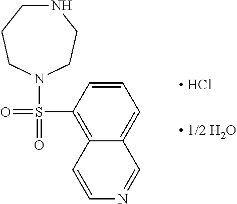 [SMILES: O=S(=O)(c1cccc2cnccc12)N1CCCNCC1 ; "· 1/2 H2O" ; "· HCl"]

### Hexahydro-1-(5-isoquinolinesulfonyl)-1H-1,4-diazepine monohydrochloride hemihydrate

Fasudil is a selective inhibitor of protein kinases, such as ROCK, PKC and MLCK and treatment results in a potent relaxation of vascular smooth muscle, resulting in enhanced blood flow (Shibuya 2001). A particularly important mediator of vasospasm, ROCK induces vasoconstriction by phosphorylating the myosin-binding subunit of myosin light chain (MLC) phosphatase, thus decreasing MLC phosphatase activity and enhancing vascular smooth muscle contraction. Moreover, there is evidence that fasudil increases endothelial nitric oxide synthase (eNOS) expression by stabilizing eNOS mRNA, which contributes to an increase in the level of the potent vasodilator nitric oxide (NO), thereby enhancing vasodilation (Chen 2013).

Fasudil has a short half-life of about 25 minutes, but it is substantially converted in vivo to its 1-hydroxy (M3) metabolite. M3 has similar, but non-identical, effects to its fasudil parent molecule in vasodilation mediated by MLCK inhibition, with slightly enhanced activity and a half-life of about 8 hours (Shibuya 2001). It is not known what is the pharmacological contribution of the parent versus the metabolite in vivo, especially regarding the activity on ROCK, the primary target of therapy according to the invention, rather than MLCK. M3 exists as two tautomers, depicted below:

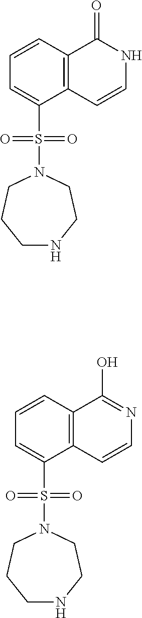 [SMILES: O=c1[nH]ccc2c(S(=O)(=O)N3CCCNCC3)cccc12 ; O=S(=O)(c1cccc2c(O)nccc12)N1CCCNCC1]

The ROCK inhibitors used in the invention, such as fasudil, include pharmaceutically acceptable salts and hydrates. Salts that may be formed via reaction with inorganic and organic acid. Those inorganic and organic acids are included as following: hydrochloric acid, hydrobromide acid, hydriodic acid, sulphuric acid, nitric acid, phosphoric acid, acetic acid, maleic acid, maleic acid, maleic acid, oxalic acid, oxalic acid, tartaric acid, malic acid, mandelic acid, trifluoroacetic acid, pantothenic acid, methane sulfonic acid, or para-toluenesulfonic acid.

### Pharmaceutical Compositions

Pharmaceutical compositions of ROCK inhibitors usable in the are generally oral and may be in the form of tablets or capsules and may be immediate-release formulations or may be controlled- or extended-release formulations, which may contain pharmaceutically acceptable excipients, such as corn starch, mannitol, povidone, magnesium stearate, talc, cellulose, methylcellulose, carboxymethylcellulose and similar substances. A pharmaceutical composition comprising a ROCK inhibitor and/or a salt thereof may comprise one or more pharmaceutically acceptable excipients, which are known in the art. Formulations include oral films, orally disintegrating tablets, effervescent tablets and granules or beads that can be sprinkled on food or mixed with liquid as a slurry or poured directly into the mouth to be washed down.

Pharmaceutical compositions containing ROCK inhibitors, salts and hydrates thereof can be prepared by any method known in the art of pharmaceutics. In general, such preparatory methods include the steps of bringing a ROCK inhibitor or a pharmaceutically acceptable salt thereof into association with a carrier or excipient, and/or one or more other accessory ingredients, and then, if necessary and/or desirable, shaping, and/or packaging the product into a desired single- or multi-dose unit.

Pharmaceutical compositions can be prepared, packaged, and/or sold in bulk, as a single unit dose, and/or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition of the invention will vary, depending upon the identity, size, and/or condition of the subject treated and further depending upon the route by which the composition is to be administered. The composition used in accordance with the methods of the present invention may comprise between 0.001% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

In certain embodiments, the pharmaceutical composition used in the methods of the present invention may comprise a diluent. Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

In certain embodiments, the pharmaceutical composition used in the methods of the present invention may comprise a granulating and/or dispersing agent. Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM), sodium lauryl sulfate, quaternary ammonium compounds, and mixtures thereof.

In certain embodiments, the pharmaceutical composition used in the methods of the present invention may comprise a binding agent. Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (VEEGUM®), and larch arabogalactan), alginates, polyethylene oxide, polyethylene glycol, inorganic calcium salts, silicic acid, polymethacrylates, waxes, water, alcohol, and/or mixtures thereof.

In certain embodiments, the pharmaceutical composition used in the methods of the present invention may comprise a preservative. Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

In certain embodiments, the pharmaceutical composition used in the methods of the present invention may comprise an antioxidant. Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

In certain embodiments, the pharmaceutical composition used in the methods of the present invention may comprise a chelating agent. Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

In certain embodiments, the pharmaceutical composition may comprise a buffering agent together with the ROCK inhibitor or the salt thereof. Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

In certain embodiments, the pharmaceutical composition used in the methods of the present invention may comprise a lubricating agent. Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

In other embodiments, the pharmaceutical composition of containing a ROCK inhibitor or salt thereof will be administered as a liquid dosage form. Liquid dosage forms for oral and parenteral administration include pharmaceutically acceptable emulsions, microemulsions, solutions, suspensions, syrups, and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates of the invention are mixed with solubilizing agents such as Cremophor™, alcohols, oils, modified oils, glycols, polysorbates, cyclodextrins, polymers, and mixtures thereof.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Some compositions of the invention relate to extended- or controlled-release formulations. These may be, for example, diffusion-controlled products, dissolution-controlled products, erosion products, osmotic pump systems or ionic resin systems. Diffusion-controlled products comprise a water-insoluble polymer which controls the flow of water and the subsequent egress of dissolved drug from the dosage from. Dissolution-controlled products control the rate of dissolution of the drug by using a polymer that slowly solubilizes or by microencapsulation of the drug-using varying thicknesses to control release. Erosion products control release of drug by the erosion rate of a carrier matrix. Osmotic pump systems release a drug based on the constant inflow of water across a semi permeable membrane into a reservoir which contains an osmotic agent. Ion exchange resins can be used to bind drugs such that, when ingested, the release of drug is determined by the ionic environment within the gastrointestinal tract.

### Methods of Treatment

### Treatable Patients

Patients treatable according to the invention will have an actual, suspected, or probable diagnosis of traumatic encephalopathy syndrome (TES). TES is a syndrome found in people with a history of repetitive brain trauma, including symptomatic concussions as well as asymptomatic subconcussive hits to the head that do not cause symptoms. According to recent standards articulated by the National Institutes of Health, a diagnosis of TES requires: (a) substantial exposure to repetitive head impacts (RHIs); (b) cognitive impairment, neurobehavioral dysregulation, or both; and (c) the deficits are not fully accounted for by other disorders and conditions.

### Substantial Exposure to Repetitive Head Impacts (RHIs)

History of substantial exposure to RHIs over the subject's lifetime are characteristic of TES. These impacts may or may not have been associated with clinical symptoms or signs of concussion or traumatic brain injury (TBI).

There are many possible sources of substantial exposure to RHIs. For example, involvement in high-contact or collision sports like boxing, American (tackle) football, ice hockey, soccer, rugby, professional wrestling, mixed martial arts (MMA), motocross, bull riding and the like. Military service may also be a source of RHIs, including combat exposure to multiple blast and other explosions, noncombat exposure to explosions (including breacher training-blasting and forced opening of locked doors), or multiple blows to the head over an extended period (e.g., pugil stick training). Domestic violence (or intimate partner violence), poorly controlled epilepsy that results in repeated trauma, head banging, and vocational activities such as breaching locked doors and other barriers by first responders may also be a source of RHIs.

The threshold for substantiality is somewhat subjective, but it is significantly more than occasional or periodic and generally occurs over an extended period of time. It will differ based on frequently a particular activity may result in a head injury. For sports like boxing or MMA, head injuries would be expected to be more frequent and so the amount of time a subject would be expected to participate in a sport and meet the threshold of substantiality would be lower than in sports such as motocross, where head injuries would be expected much less frequently. For American football, as an example, generally, a minimum of 5 years of organized play is a guideline, including ≥2 year at the high school level or beyond. Involvement in other high-risk activities should be likewise of substantial duration. Thus, substantiality typically would require participation in one or more risky activities for at least 2 years, but generally 3 to 4 years and preferably at least 5 years. The diagnostic guidelines allow for clinical judgment by the physician, which is well within the ability of the skilled clinician who will be guided by the frequency and severity of RHIs because the syndrome is related to the cumulative damage. Thus, the higher the frequency and severity, the fewer years of participation would be expected before the syndrome emerges.

### Core Clinical Features: Cognitive Impairment, Neurobehavioral Dysregulation

A progressive course of cognitive impairment, neurobehavioral dysregulation, or both, are indicative of TES. TES can progress through a series of stages including

Stage 1: Short-term memory loss; mild aggression and depression; headaches.

Stage 2: Severe depression, outbursts, and mood swings.

Stage 3: Aggression; apathy; memory loss; lack of spatial awareness; executive dysfunction; cognitive impairment.

Stage 4: More severe Stage 3 symptoms including cognition problems; paranoia; difficulty with language and muscle movement.

Cognitive impairment, representing a significant decline from baseline functioning, should be reported by the patient, a caregiver or a clinician. This impairment includes deficits in episodic memory and/or executive functioning. Impaired performance typically is substantiated on formal neuropsychological testing, if available. TES is indicated by a performance at a level of at least 1.5 standard deviations below appropriate norms, accounting for the individual's estimated premorbid functioning. If formal neuropsychological testing is not available, there should be evidence of impairment below expected norms and/or a person's estimated baseline in episodic memory and/or executive functioning on a standardized mental status examination (e.g., Montreal Cognitive Assessment and Mini-Mental State Examination) by a practitioner experienced in the evaluation of cognition.

Neurobehavioral dysregulation should be reported by the patient, an informant or assessed by a clinician. It represents a significant change from baseline functioning, which may require clinical judgment in cases where the baseline is shifting during the course of ongoing RHI exposure.

Symptoms and/or observed behaviors generally represent poor regulation or control of emotions and/or behavior. These may include, but are not limited to, explosiveness, impulsivity, rage, violent outbursts, having a short fuse (more than periodic episodes of minor irritability), or emotional lability (mood swings). Standard measures are available that can be used to demonstrate clinical impairment in these domains, but these will not be available in many cases. These symptoms and/or observed behaviors will represent an obvious change from baseline and do not appear to represent a transient response to life events, e.g., divorce, death of loved one, and financial problems.

The following table presents a list of common cognitive tests with which one skilled in the are will be familiar and which are helpful in both diagnosing TES and in assessing the outcomes of the inventive methods of treatment.

### Common Cognitive Tests

| | |
|---|---|
| Test | Description |

Processing Speed

| | |
|---|---|
| Pattern Comparison Processing | Determine whether a set of two pictures placed side by |
| Speed Test | side are the same for 90 seconds. |
| Digit Symbol Substitution Test | Timed measure consisting of nine digit-symbol pairs; |
| | participant matches the correct symbol to a list of digits. |

Sustained attention

| | |
|---|---|
| Connors Continuous Performance | Measure selective response to target inputs while doing a |
| Test | repetitive task -- for example, respond only when they will |
| | see or hear the number 1, but not the number 2. |

Selective attention

| | |
|---|---|
| Target Letter Search | Search a visual display for a target, such as a letter, that is |
| | surrounded by other, non-target letters. |
| Stroop Test | Name the color of ink in which another color word is |
| | printed (e.g., the word “red” printed in the color green). |

Episodic memory

| | |
|---|---|
| Picture Sequence Memory Test | Recall series of 6 to 18 illustrated objects and activities |
| | presented in a certain order. |

Working memory

| | |
|---|---|
| General | Common tests include giving participants a list of items |
| | (letters, numbers, words) and asking them to repeat the |
| | items back in order. |
| List Sorting Test | Participants see pictures and written names of foods and |
| | animals and hear audio names. The participant recites |
| | them back in order of size from smallest to largest, first |
| | within a single category (e.g., animals) and then in two |
| | categories (e.g., foods, then animals). |

Semantic memory

| | |
|---|---|
| Category Fluency Test | Name all of the items they can in a category (e.g., animals) |
| | in 1 minute. |
| Boston Naming Test | Subject sees a series of pictures and must name each |
| | picture within 20 seconds |

Executive function

| | |
|---|---|
| Wisconsin Card Sorting Test | Subject matches two sets of cards according to some |
| | characteristic, but the participant is not told the rules - |
| | they learn through feedback. After a fixed number of |
| | correct matches, the rules are changed without notice, and |
| | the new rule must be learned. |
| Trail Making Test | Subject connects 25 consecutive numbers or alternating |
| | numbers and letters in ascending order. |

Reasoning

| | |
|---|---|
| Letter Sets Test | consists of five sets of four letters. The participant has to |
| | decide which one of the five sets is dissimilar in the sense |
| | that it does not follow a rule used to generate the other four |
| | sets. Tests of inference are used to measure the ability to |
| | reason and draw conclusions from information presented |
| | in statements. |

Language

| | |
|---|---|
| General | There are a variety of tests, depending on the particular |
| | aspect of language being assessed. These include a word- |
| | by-word reading paradigm to assess comprehension of |
| | sentences (e.g., Kemtes and Kemper, 1997), word-by- |
| | word reading time (e.g., Stine, 1990) or recall of text |
| | (Stine-Morrow et al., 1996). |
| Token Test | Subject presented with tokens that vary in shape color and |
| | size and must follow commands related to identifying |
| | them (e.g., touch the circle). |
| Boston Naming Test | Confrontational naming test - subject shown objects to |
| | name in certain period of time. |
| Category Fluency Test | Subject is asked to name items in a category, like words |
| | beginning with F, A and S, or as many animals as possible |
| | in a particular time. |
| Boston Diagnostic Aphasia | A testing battery that evaluates language skills based on |
| Examination | perceptual modalities (auditory, visual, and gestural), |
| | processing functions (comprehension, analysis, problem- |
| | solving), and response modalities (writing, articulation, |
| | and manipulation). |

Spatial ability

| | |
|---|---|
| Paper Folding Test | Decide which pieces of paper with holes punched in them |
| | is the same by looking at them folded and unfolded. |
| Mental Rotations Test | Compare several three-dimensional objects that are |
| | rotated differently and state whether they are the same or |
| | mirror images. |
| Block Design Test | Subtest of the Wechsler Adult Intelligence Scale (WAIS). |
| | Arrange a set of 3-dimensional blocks to match a 2- |
| | dimensional drawing using color cues within a specific |
| | time limit. |

Intelligence

| | |
|---|---|
| Wechsler Adult Intelligence Scale | Consists of 10 core subtests and 5 supplemental subtests. |
| | The core subtests provide the intelligence quotient, which |
| | is derived from four index scores: Verbal Comprehension |
| | Index, Perceptual Reasoning Index, Working Memory |
| | Index, and Processing Speed Index. |

The ADAS-COG (Alzheimer's Disease Assessment Scale-cognitive) is a particularly useful global assessment tool that assesses the following domains using this scoring system, resulting in a score of between 0 and 150, indicating the number of errors made in each assessment. Higher scores indicate greater deficits.

| Task | Description | Scoring |
|---|---|---|
| Word Recall | A list of 10 words is read by | Mean number of words not |
| | the subject, and then the | recalled across the three trials; |
| | subject is asked to verbally | scoring range is 0 to 10. |
| | recall as many of the words as | |
| | possible. Three trials of | |
| | reading and recalling are | |
| | performed. | |
| Naming Objects and Fingers | The subject is asked to name | The number of fingers and |
| | the fingers of their dominant | objects correctly named; |
| | hand as well as twelve objects: | scoping range is 0 to 4. |
| | flower (plastic), bed (doll | |
| | house furniture), whistle, | |
| | pencil, rattle, mask, scissors, | |
| | comb, wallet, harmonica, | |
| | stethoscope, and tongs. | |
| Commands | The subject is asked to | Scored from 0 to 5 based on |
| | perform commands that | the largest number of steps |
| | involve one to five steps. For | that are correctly performed |
| | example, the two-step | (score is 0 if five step |
| | command is to “Point to the | command is correctly |
| | ceiling, then to the floor.” | performed). |
| Constructional Praxis | The subject is shown four | Scored from 0 to 5 based on |
| | geometric forms (circle, two | the number of correctly drawn |
| | overlapping rectangles, | forms. |
| | rhombus, cube) and asked to | |
| | copy them on a piece of paper. | |
| Ideational Praxis | The subject is asked to pretend | Scored from 0 to 5 based on |
| | to send a letter to themselves: | difficulty of performing the |
| | fold letter, put letter in | five components. |
| | envelope, seal envelope, | |
| | address envelope, and put a | |
| | stamp on the envelope. | |
| Orientation | The subject is asked the date, | The number of correct |
| | month, year, day of the week, | responses; scoring range is 0 |
| | season, time of day, place, and | to 8. |
| | person. | |
| Word Recognition | The subject reads twelve | Mean number of correct |
| | words aloud, and then these | responses across the three |
| | twelve words are randomly | trials; scoring range is 0 to 12. |
| | shuffled with twelve new | |
| | words, and the subject is asked | |
| | whether they have previously | |
| | seen each of the twenty-four | |
| | words. Three trials are | |
| | performed. | |
| Language | After the administration of the | Quality of speech is given a |
| | Word Recall task (Q1) ten | global rating by the |
| | minutes of open-ended | administrator that ranges from |
| | conversation occur between | 0 to 5. |
| | the test administrator and | |
| | subject, before the remainder | |
| | of the tasks are presented. | |
| | These ten minutes of | |
| | conversation are used to | |
| | assess language ability. | |
| Comprehension of Spoken | This task also relies on the ten | The administrator provides a |
| Language | minutes of open-ended | score from 0 to 5. |
| | conversation. The | |
| | administrator provides an | |
| | assessment of how well the | |
| | subject can understand | |
| | speech. | |
| Word Finding Difficulty | During the aforementioned | The administer provides a |
| | open-ended conversation, the | score from 0 to 5. |
| | administrator assesses how | |
| | much difficulty the subject has | |
| | in finding desired words. | |
| Remembering Test | The administrator provides an | The administrator provides a |
| Instructions | assessment according to the | score from 1 to 5. |
| | number of times that the | |
| | subject needed to be reminded | |
| | of instructions for the Word | |
| | Recognition task. | |

Many general assessment tools are available to assess neurobehavioral deficits and these include the Patient Health Questionnaire-9 (PHQ-9), Neurobehavioral Core Test Batter (NCTB), Neurobehavioral Evaluation System (NES), Neuropsychiatric inventory questionnaire (Depression and Neuropsychiatric conditions), the Alzheimer's Disease Assessment Scale Non-Cognitive (ADAS-Noncog), the Behavior Rating Scale for Dementia of the Consortium to Establish a Registry for Alzheimer's Disease (CERAD-BRSD), and the Behavioral Pathology in Alzheimer's Disease Scale (BEHAVE-AD).

The degree of depressive symptomology was deemed to be a predictor of TES diagnosis in retired athletes. (Schaffert 2021). If a certain domain is identified and more sensitivity is desired, more specific scales known to one of skill in the art include the Geriatric Depression Scale: Short Form (Depression), Beck's depression inventory (Depression), Columbia suicide severity score (Suicidality), Hamilton depression scale (Depression), Hospital anxiety and depression scale (Anxiety and depression), the Cornell Scale for Depression in Dementia (Depression) and the Dementia Mood Assessment Scale (DMAS; Depression), and the Cohen-Mansfield Agitation Inventory (CMAI; Agitation symptoms).

TES is indicated where there is progressive worsening of these clinical features, as measured by any of the foregoing cognitive and non-cognitive assessments over at least 1 year in the absence of continued exposure to RHIs or TBI. Such worsening may be evidence on a full assessment scale and/or on a specific domain of one or more assessment.

### Ruling Out Other Disorders

For a TES diagnosis, the pattern of the cognitive deficits or the core clinical feature of neurobehavioral dysregulation will not be fully accounted for by other preexisting, established, or acquired nondegenerative nervous system, medical, or psychiatric disorders and conditions. To be clear, a comorbid diagnosis of another neurodegenerative disease does not exclude a TES diagnosis. Any finding based on clinical features, any available biomarkers or other evidence that is fully accounted for by another neurodegenerative disorder can support a TES diagnosis. Comorbid diagnosis, for example, of substance use disorder, post-traumatic stress disorder, mood or anxiety disorders, or a combination of these can be present and do not exclude a TES diagnosis, unless they are determined to account for all core clinical features.

Imaging is as useful tool in diagnosing TES and especially in possibly ruling out other neurological or neurodegenerative conditions. Particularly useful are computerized tomography (CT), magnetic resonance imaging (MRI) and positron emission spectroscopy (PET). These tools can be sued to look at overall brain volume and can be used to detect the regional losses in volume that are characteristic of non-TES conditions. Automated tools are increasingly available that can perform these functions.

Fluorodeoxyglucose (FDG) PET scans measure glucose use in the brain. Glucose, a type of sugar, is the primary source of energy for cells. Studies show that people with dementia often

have abnormal patterns of decreased glucose use in specific areas of the brain. An FDG PET scan can show a pattern that may support a diagnosis of dementia versus TES.

Amyloid PET scans measure abnormal deposits of beta-amyloid. While amyloid plaques also occur in the normal aging brain, higher levels of beta-amyloid are consistent with Alzheimer's disease. Several tracers may be used for amyloid PET scans, including florbetapir, flutemetamol, florbetaben, Pittsburgh compound B and NAV4694.

Non-neurodegenerative conditions that can be found to meet the TES symptom criteria included those with mood disorder, and/or chronic pain and those with suicidal ideation.

### Level of Functional Dependence

Following the diagnosis of TES, the severity of the syndrome is graded according to the level of functional dependence, which is based on the impact of cognitive impairment and/or neurobehavioral dysregulation and not on physical limitations or medical illness.

Independent: The independent patient will be able to function at a normal level at work, with household responsibilities, and in family, social, and community roles. They are able to engage in hobbies and intellectual activities at usual levels. They score normally/independent on scales that measure activities of daily living (ADLs). TES patients that are independent generally will not be treated in accordance with the invention.

Mild functional limitation: These patients have slightly reduced performance on the job, in household responsibilities, or family, social and community roles. They may have slight problems in hobbies and intellectual interests. The rate as mostly independent but may be more challenged in some instrumental ADLs (e.g., managing money, cleaning and maintaining the house, preparing meals, shopping for groceries and necessities, transportation within the community, medication management, using the telephone) and fully independent in basic ADLs (e.g., personal hygiene and grooming, toilet hygiene, bathing/showering, dressing, self-feeding, and functional mobility). Patients with mild functional limitations may be treated in accordance with the invention.

Patients preferably treated according to the invention will have greater degrees of functional impairment and will generally be considered to have dementia. While dementia can be assessed using broad cognitive assessments like the MoCA (score 25 or lower) or the MMSE (score 23 or lower), a patient is typically considered to have dementia based on a cognitive deficit that causes a functional impairment.

Mild dementia: These patients have a clear impairment of instrumental ADLs. They may be engaged in some home, family, social, and community activities. They no longer participate in more difficult activities and need cues to engage in some basic ADLs.

Moderate dementia: These patients are not independent but can be taken to some functions outside the home. They can only perform simple chores, have very restricted interests and need assistance with basic ADLs.

Severe dementia: Cannot participate in functions outside the home; no significant function in home; impaired basic ADLs; not independent with self-care; frequently incontinent.

Patients treatable according to the invention will generally display some level of functional limitation and many patients will be diagnosed with dementia.

### Age

While TES may manifest at nearly any age, it has, surprisingly, been found that rho kinase inhibitor treatment is more effective in a younger population. Accordingly, preferred methods contemplate treating patients 75 years old or younger, while more preferred methods contemplate treating patients 70 years old or younger, while some methods contemplate treating patients 65 years or younger.

### Dosing

In accordance with the treatment methods of the present invention, a therapeutically effective amount of a ROCK inhibitor or a pharmaceutically acceptable salt thereof is administered to a TES patient one or more times a day. The lowest therapeutically effective amount of fasudil, for example, is 60 mg per day, generally administered in 2 to 3 equal portions to obtain the full daily dose. The highest therapeutically effective dose may be determined empirically as the highest dose that remains effective in alleviating one or more TES symptoms, but does not induce an unacceptable level or adverse events. Fasudil, for example, generally will not be administered in a daily dose exceeding 240 mg.

One preferred dosing regimen involves the treatment with 60 mg of fasudil hydrochloride hemihydrate three times per day using an oral immediate-release formulation, for a total daily dose of 180 mg. Other daily doses will range from 90 mg to 180 mg per day b.i.d. A further dosing regimen involves the treatment with 90 mg of fasudil hydrochloride hemihydrate only two times per day using an immediate-release formulation, for a total daily dose of 180 mg. In other embodiments, fasudil hydrochloride may be administered once a day using an immediate-release formulation at 180 mg or 240 mg. Above 240 mg per day, kidney effects of the drug are generally unacceptable. Based on ROCK inhibitory activity, one skilled in the art can readily extrapolate the provided dosing ranges for fasudil to other ROCK inhibitors.

Another embodiment involves the treatment with 90-240 mg of fasudil hydrochloride hemihydrate once per day in an extended release dosage form. Treatment with an extended release total daily dose of 180 mg fasudil hydrochloride hemihydrate is preferred. Generally an extended release dosage form will contain between 180 and 240 mg of fasudil hydrochloride hemihydrate.

Methods of administering compositions according to the invention would generally be continued for at least one day. Some preferred methods treat for up to 30 days or up to 60 days or even up to 90 days or even more. Treatment for more than 60 days is preferred and treatment for at least 6 months is particularly preferred. The precise duration of treatment will depend on the patient's condition and response to treatment. Most preferred methods contemplate that treatment begins after the onset or appearance of symptoms.

It will be appreciated that dose ranges as described herein provide guidance for the administration of provided pharmaceutical compositions to an adult. The amount to be administered to, for example, a child or adolescent can be determined by a medical practitioner or person skilled in the art and can be lower or the same as that administered to an adult.

Methods of administering compositions according to the invention would generally be continued for at least one day. Some preferred methods treat for up to 30 days or up to 60 days or even up to 90 days or even more. Treatment for more than 60 days is preferred and treatment for at least 6 months is particularly preferred. The precise duration of treatment will depend on the patient's condition and response to treatment.

### Assessing Outcomes of Treatment

The results of the inventive treatment regiments may be measured using the cognitive and neurobehavioral scales used in the diagnosis of TES. It may also be assessed using more general cognitive scales, such as the Montreal Cognitive Assessment (MoCA) and the Mini-Mental State Exam (MMSE).

The MoCA is a one-page 30-point test that takes approximately 10 minutes to administer. It measures the following cognitive domains. Short-term memory where the subject learns 5 nouns in two trials and then is asked to recall them after 5 minutes. Visuospatial abilities are assessed using a clock-drawing task (3 points) and a three-dimensional cube copy (1 point). Executive function is assessed using an alternation task (variation the trail-making task) (1 point), a phonemic fluency task (1 point), and a verbal abstraction task (2 points). Attention, concentration, and working memory are evaluated using a sustained attention task (1 point), a serial subtraction task (3 points), and digits forward and backward (1 point each). Language is assessed using a three-item confrontation naming task (lion, camel, rhinoceros; 3 points), repetition of two syntactically complex sentences (2 points), and the phonemic fluency task, mentioned above. Abstract reasoning is assessed using a describe the similarity task (2 points). Finally, orientation to time and place is evaluated by asking the subject for the date and the city in which the test is occurring (6 points). Scores range between 0 and 30. A score of 26-30 is considered normal and a score of less than 26 is considered cognitively impaired. Accordingly, subjects treatable according to the invention preferably have a MoCA score of 25 or less. The MoCA is believed by certain people to be very difficult, especially the last five questions.

The MMSE, is described fully in Folstein (1975, 1987 and 2007) and is very similar to the MoCA. An MMSE score of 24-30 indicates no cognitive impairment and a score or less is

considered to be cognitively impaired. Accordingly, subjects treatable according to the invention preferably have a MMSE score of 23 or less.

Treatment using the inventive methods generally result in improved cognitive functioning on general tests of cognition like the MoCA or the MMSE and/or on cognitive tests directed to one or more cognitive domains. The inventive treatment methods may also improve neurobehavioral symptoms using one or more of the aforementioned neurobehavioral assessment; such improvement be independent of cognitive improvement or may occur along with cognitive improvement. Scores or times (as appropriate for the test) will generally improve by a minimum of 10% with treatment as compared to without treatment. Thus, MoCA and/or MMSE scores will improve by at least 2 points and generally by at least 3 points with drug treatment. In addition, subjects treated with drug will show reduced rate of cognitive decline than those who are not treated, as measure by the MoCA, the MMSE and/or one or more specific cognitive tests. In some embodiments, improvements in scores or times will be a minimum of 20% on treatment as compared to without treatment. In a preferred embodiment, treatment of a patient with fasudil reduces or reverses the progression of TES by one or more of the foregoing cognitive measures.

The methods of the invention also contemplate administering ROCK inhibitors with other compounds used to improve cognition. They may be administered in combination, a single dosage form, in a common dosing regimen or administered to the same patient at different times of the day using different dosing regimens.

### Example 1

A clinical trial was conducted to determine the effectiveness of oral fasudil in treating patients with Alzheimer's Dementia (AD).

Nineteen patients with Alzheimer's Dementia were recruited and treated for 6-12 weeks with fasudil HCl hemihydrate. All the patients were treated for 6 weeks with 90 mg per day (30 mg TID) and a subset of patients was treated an additional 6 weeks with 180 mg per day (60 mg TID). Both treatment regimens were well tolerated.

Fourteen subjects completed the two blinded portions of the study. A variety of assessments were conducted at baseline and at the end of treatment, including the mini mental state exam (MMSE). The MMSE is an 11-question measure that tests five areas of cognitive function: orientation, registration, attention and calculation, recall, and language. The MMSE has a maximum score of 30. Scores of 24-30 are considered cognitively normal, 19-23 is considered mild dementia and scores of 10-18 considered moderate dementia and below 10 is severe dementia.

Each patient has a pre-treatment baseline score, an open-label treatment score (at the end of the open-label period), a double-blind placebo score (end of the blinded placebo period) and a double-blind drug treatment score (end of the blinded drug treatment period). If the drug is having an effect on a patient, the expectation is that when the patient is on drug, the MMSE should increase and then when the patient is on placebo it should decrease. Thus, MMSE would be expected to increase from baseline in the open-label period, be relatively lower in the placebo double blind period and be relatively higher in the drug double blind period.

Only three patients showed an expected change each time the treatment assignment was changed between active and placebo, patient 7001-7, 9005-23 and 2006-21, though the effect is most pronounced in 7001-7. Patient 7001-7 started with an MMSE of 18 (moderate dementia), the MMSE increased to 30 (cognitively normal) at the end of the open-label period. The next period was placebo and the patient's MMSE declined to 23 (mild dementia). Finally, the patient was treated with drug again and the MMSE again increased to 29 (cognitively normal). Patient 9005-23 started with an MMSE of 9 (severe dementia), which increased to 11 (moderate dementia) on drug, decreased to 8 (severe dementia) on placebo and increased to 12 (moderate dementia) on drug. Patient 2006-21 had a baseline MMSE of 12 (moderate dementia) and improved to 15 (moderate dementia) on drug, declined to and MMSE of 14 (moderate dementia) on placebo and then improved again to an MMSE of 17 (moderate dementia) on drug in the blinded portion of the study.

Because the patient showed such a dramatic and consistent effect on drug treatment, patient 7001-7 was selected for further assessment. The patient, caregiver and all medical staff were blinded to treatment assignment and there is no evidence than anyone was unblinded. Reviewing the patient's medical history, recounted below, there was nothing, other than the patient's relatively young age, that suggested anything unique.

Subject 7001-7 is a 60-year-old Caucasian male with early onset Alzheimer's disease. He has Type 1 Diabetes Mellitus and was treated with insulin. He has coronary artery disease, having a had a bypass, and stent placements. He has atrial fibrillation and is treated with dronedarone, nitroglycerin, metoprolol and aspirin. He has hypocholesteremia and is treated with atorvastatin. Alzheimer's Disease was diagnosed in 5 years prior to entering the study and is treated with memantine and rivastigmine. He also has a history of GERD and is treated with famotidine and pantoprazole. He has depression, anxiety, and reduced appetite and is treated with fluoxetine and buspirone. He experiences headaches and lightheadedness and was vaccinated against COVID and pneumonia. Despite the relatively young age, the subject has never been assessed using PET for beta-amyloid.

The patient's MRI scans were sent to an independent expert in dementia with the foregoing medical history; the expert was asked to evaluate whether there was anything unusual. He found no indications on the MRI of either Alzheimer's Disease or vascular dementia. The latter was particularly surprising in view of the medical history which has extensive pre-existing cardiovascular disease. The only clear abnormality he noted was cavum septum, something not mentioned in the report in the accompanying MRI report. Cavum septum, something is commonly seen in boxing, soccer and football players and is associated with CTE.

With this finding, the subject's wife was queried as to his history playing sports and other history of concussions. She revealed that he played football throughout high school, playing defensive end. In one notable incident, he broke his jaw when a linebacker hit him in the face with his helmet; he missed the rest of the season. He was also on the ski team.

Over two decades ago he climbed a ladder to access the roof and it slipped off the deck, went through the railing and he fell/tumbled about 40 feet down, over a cliff. He had blood coming out of his ear and nose and he seemed dazed. He had a broken shoulder and a hairline fracture to his back, but they were not advised of any head injuries.

It was concluded that the subject was most likely suffering from TES and further assessments are expected to confirm this diagnosis.

Overall, improvements on all domains measured were more pronounced in the younger population (70 years old or younger), suggesting there is generally a greater degree of plasticity in the younger population or perhaps a common mechanism of injury, such as TES. No patients were formally diagnosed in the study and it is possible that the younger population were all subjects with TES and not AD. Each of the references cited are herein incorporated by reference in their entireties.

1. A method of treating traumatic encephalopathy syndrome (TES) in a patient in need of treatment, comprising administering to a subject diagnosed with TES a therapeutically effective amount of a rho kinase inhibitor.

2. The method of claim 1, wherein the patient is suffering from a cognitive deficit.

3. The method of claim 1 or 2, wherein the patient has a history of repetitive head impacts as a result of playing American football.

4. The method of any one of claims 1-3, wherein the patient is 70 years old or younger.

5. The method of any one of claims 1-4, wherein the patient has cavum septum.

6. The method of any one of claims 1-5, wherein the patient has dementia.

7. The method of any one of claims 1-6, wherein the patient has moderate dementia.

8. The method of any one of claims 1-7, wherein the patient is suffering from a neurobehavioral deficit.

9. The method of any one of claims 1-8, wherein the rho kinase inhibitor is an isoquinoline derivative.

10. The method of any one of claims 1-9, wherein the isoquinoline derivative is fasudil, a salt, or a derivative thereof.

11. The method of any one of claims 1-10 where said treatment continues for at least 6 weeks.

12. The method of any one of claims 1-11, wherein said dose is administered in three equal portions throughout the day.

13. The method of any one of claims 1-12, wherein the total daily dose is between 70 mg and 240 mg.
